(19) United States
(12) Patent Application Publication
(10) Pub. No.: US 2006/0194263 A1
(43) Pub. Date: Aug. 31, 2006

(54) Small molecule mediated, heterogeneous, carbon nanotube biosensing

(76) Inventors: Salah Boussaad, Wilmington, DE (US); Bruce A. Diner, Chadds Ford, PA (US); Janine Fan, Hockessin, DE (US); Vsevolod Rostovtsev, Swarthmore, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/241,368

(22) Filed: Sep. 30, 2005

Related U.S. Application Data
(60) Provisional application No. 60/615,060, filed on Sep. 30, 2004.

(51) Int. Cl. G01N 33/542; C12Q 1/54; C12M 1/34

(52) U.S. Cl. 435/7.9; 435/287.2; 435/14; 205/777.5; 977/702

(57) ABSTRACT

Nanosensors for detecting ananlytes and methods of detecting analytes have been developed in which a small molecule effector concentration is altered thereby causing changes in carbon nanotube conductance.

### FIELD OF INVENTION

This invention relates to the field of nanotechnology. Specifically the invention describes a nanosensor for the detection of an analyte in which a small molecule effector concentration is altered thereby causing changes in carbon nanotube conductance.

### BACKGROUND OF THE INVENTION

There is an increasing need for rapid, small scale and highly sensitive detection of biological molecules in medical, bioterrorism, food safety, and research applications. Nanostructures such as silicon nanowires and carbon nanotubes display physical and electronic properties amenable to use in miniature devices. Carbon nanotubes (CNTs) are rolled up graphene sheets having a diameter on the nanometer scale and typical lengths of up to several micrometers. CNTs can behave as semiconductors or metals depending on their chirality. Additionally, dissimilar carbon nanotubes may contact each other allowing the formation of a conductive path with interesting electrical, magnetic, nonlinear optical, thermal and mechanical properties.

It is known that single-walled carbon nanotubes are sensitive to their chemical environment, specifically that exposure to air or oxygen alters their electrical properties (Collins et al.(2000) Science 287:1801). Additionally, exposure of CNTs to gas molecules such as NO₂ or NH₃ alters their electrical conductance (Kong et al. (2000) Science 287:622). Thus chemical gas sensors can be designed on the basis of the electrical properties of carbon nanotubes such as described in DE10118200.

Detection of biomolecules has been achieved using probes that are attached to nanotubes or silicon nanowires. For example, a device using peptide nucleic acid receptors designed to recognize a specific DNA sequence and attached to the surface of silicon nanowires was able to detect the presence of a DNA sequence through hybridization-induced conductance changes (Hahm and Lieber (2003) Nano Lett. 4:51). Hybridization of a single-stranded DNA probe attached to silicon nanowires with the complementary DNA strand was detected by conductance changes (Z. Li et al. (2003) Nano Lett. 4:245). In these two cases detection depends on the nanowires behaving as field effect transistors where changes in nanowire conductance result from binding of the target DNA to its complement directly at the nanowire surface.

Hybridization of a single-stranded polyC DNA probe attached to carbon nanotubes with the complementary polyG DNA strand was detected amperometrically. (J. Li et al. (2003) Nano Lett. 3:597). In this case the oxidation of Ru(bpy)₃²⁺ was mediated by the guanine bases of the DNA that is attached to the CNT.

In WO 02/48701, articles are described that use nanowires, including CNTs, to detect different types of analytes including biological analytes. The nanowire may be modified by attaching an agent that is designed to bind an analyte, the binding to the nanowire or to a coating on the nanowire then causes a detectable change in conductance. In this detection system, the interaction between the binding agent and the analyte to be detected alters the electrical conductance of the nanowire. This requirement in turn limits the functional location of the binding agent with respect to the nanowire in that they must be in close proximity, 5 nanometers or less.

There is a need for a nanoscale detection system that does not require the binding of a binding agent and target analyte directly to the detecting nanowire or CNT. Applicants have solved this problem by developing a nanosensor that responds to a target analyte by producing or consuming, at a distance, an effector that causes a change in the electrical conductance of the CNT. In addition to expanding the possibility of binding agent-target analyte pairs that may be used in a nanoscale detection system, this novel system removes the spatial limitation for close proximity of the binding agent and CNT.

### SUMMARY OF THE INVENTION

The present invention provides a nanosensor for the detection of an analyte. In a first embodiment the nanosensor uses electrically conducting paths of semiconducting single-walled carbon nanotubes to sense changes in concentration of an effector molecule. Alterations in the concentration of the effector molecule alter the conductance of the CNTs relative to a baseline conductance, thereby producing a quantifiable signal that can be correlated to the presence of the analyte. The concentration of the effector is altered in the presence of the analyte by a reporter, typically as part of a reporter conjugate, acting on a reporter substrate where the presence of the analyte causes the reporter conjugate to be attached to a surface.

In a second embodiment, a capture moiety on a surface captures a catalytically active analyte, the presence of which is detected by the action of the analyte on a reporter substrate which in turn alters the effector concentration.

In a third embodiment a reporter molecule is attached to a surface and a reaction between the analyte and the reporter molecule causes a change in the concentration of the effector. The invention also provides methods for detecting an analyte through the detection of an effector whose concentration is altered in the presence of a reporter molecule, or of a reporter substrate. In all of the above mentioned embodiments, the change in the effector concentration is amplified such that its change in concentration exceeds that of the analyte.

Optionally, the reporter or the reporter conjugate may be provided in inactive form and be controlled by an activity switch which effects reporter activation upon binding by the reporter conjugate to the analyte. Accordingly the invention provides a nanosensor for detecting the presence of an analyte comprising:
a) at least two electrodes connected by an electrically conducting path comprised of one or more carbon nanotubes wherein at least one of said carbon nanotubes is semiconducting and wherein the carbon nanotube is in contact with an effector;
b) a capture moiety having affinity for an analyte and attached to a surface;
c) a reporter conjugate comprising a reporter molecule linked to an analyte receptor, said analyte receptor having affinity for the analyte; and
d) a reporter substrate.

In an alternated embodiment the invention provides a nanosensor for detecting the presence of an analyte comprising:
a) at least two electrodes connected by an electrically conducting path comprised of one or more carbon nanotubes wherein at least one of said carbon nanotubes is semiconducting and wherein the carbon nanotube is in contact with an effector;
b) a capture moiety having affinity for a catalytic analyte and attached to a surface; and
c) a reporter substrate.

Similarly the invention provides a nanosensor for detecting the presence of an analyte comprising:
a) at least two electrodes connected by an electrically conducting path comprised of one or more carbon nanotubes wherein at least one of said carbon nanotubes is semiconducting and wherein the carbon nanotube is in contact with an effector; and
b) a reporter molecule attached to a surface.

In another embodiment the invention provides a nanosensor for the detection of an analyte comprising:
a) at least two electrodes connected by an electrically conducting path comprised of one or more carbon nanotubes wherein at least one of said carbon nanotubes is semiconducting and wherein the carbon nanotube is in contact with an effector;
b) a means for altering the concentration of said effector in response to the presence of an analyte.

In one aspect a method of the invention comprises a method for detecting an analyte comprising:
a) providing a nanosensor comprising:
   i) at least two electrodes connected by an electrically conducting path comprised of one or more carbon nanotubes wherein at least one of said carbon nanotubes is semiconducting, and wherein the carbon nanotube is in contact with an effector molecule and has a baseline conductance;
   ii) a capture moiety having affinity for an analyte, the capture moiety attached to a surface; and
   iii) a reporter conjugate comprising an analyte receptor and a reporter molecule;
b) providing a sample suspected of containing an analyte;
c) contacting the sample of (b) with the capture moiety of the nanosensor of (a) wherein the analyte present in the sample binds to the capture moiety and the analyte receptor of the reporter conjugate to form a capture-analyte-reporter complex;
d) contacting the capture-analyte-reporter complex of step (c ) with a reporter substrate wherein the concentration of the effector is altered resulting in a change in the conductance of the carbon nanotube with respect to the baseline conductance; and
e) measuring the change in the conductance of the carbon nanotube with respect to the baseline conductance whereby the presence of the analyte is detected.

In an alternate embodiment the invention provides a method for detecting a catalytic analyte comprising:
a) providing a nanosensor comprising:
   i) at least two electrodes connected by an electrically conducting path comprised of one or more carbon nanotubes wherein at least one of said carbon nanotubes is semiconducting, and wherein the carbon nanotube is in contact with an effector molecule and has a baseline conductance; and
   ii) a capture moiety having affinity for an analyte, the capture moiety attached to a surface;
b) providing a sample suspected of containing an catalytic analyte;
c) contacting the sample of (b) with the capture moiety of the nanosensor of (a) wherein the catalytic analyte present in the sample binds to the capture moiety to form a capture-analyte complex;
d) contacting the capture-analyte complex of step (c ) with a reporter substrate wherein the concentration of the effector molecule is altered resulting in a change in the conductance of the carbon nanotube with respect to the baseline conductance; and
e) measuring the change in the conductance of the carbon nanotube with respect to the baseline conductance whereby the presence of the analyte is detected.

Similarly the invention provides A method for detecting an analyte comprising:
a) providing a nanosensor comprising:
   i) at least two electrodes connected by an electrically conducting path comprised of one or more carbon nanotubes wherein at least one of said carbon nanotubes is semiconducting, and wherein the carbon nanotube is in contact with an effector molecule and has a baseline conductance; and
   ii) a reporter molecule having an analyte substrate, the reporter molecule attached to a surface;
b) providing a sample suspected of containing an analyte substrate wherein the concentration of the effector molecule is altered resulting in a change in the conductance of the carbon nanotube with respect to the baseline conductance; and
c) measuring the change in the conductance of the carbon nanotube with respect to the baseline conductance whereby the presence of the analyte is detected.

In a separate embodiment the invention provides a method for detecting the presence of an analyte comprising:
a) providing a nanosensor comprising:
   i) at least two electrodes connected by an electrically conducting path comprised of one or more carbon nanotubes wherein at least one of said carbon nanotubes is semiconducting and wherein the carbon nanotube is in contact with an effector and wherein the carbon nanotube has a baseline conductance;
   ii) a means for altering the concentration of said effector in response to the presence of an analyte;
b) contacting the nanosensor of (a) with an analyte whereby the concentration of said effector is altered resulting in a change from the baseline conductance of said carbon nantube; and
c) measuring the alteration from the baseline conductance of the carbon nanotube of (b) wherein the presence of the analyte is detected.

### BRIEF DESCRIPTION OF THE FIGURES AND SEQUENCES

The invention can be more fully understood from the following detailed description and the accompanying sequence descriptions, which form a part of this application.

The sequence descriptions and content of the sequence listing attached hereto (additionally provided in a computer readable form) are incorporated by reference as a part of this application. The Sequences and Sequence Listing comply with the rules governing nucleotide and/or amino acid sequence disclosures in patent applications as set forth in 37 C.F.R. §1.821-1.825 (“Requirements for Patent Applications Containing Nucleotide Sequences and/or Amino Acid Sequence Disclosures—The Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

SEQ ID NO:1 is oligo 27 and oligo 28 with sequence derived from the LDLR gene.

SEQ ID NO:2 is oligo 29 with sequence complementary to SEQ ID NO:1.

SEQ ID NO:3 is oligo 51 and oligo 53.

SEQ ID NO:4 is oligo 52.

SEQ ID NO:5 is oligo 50 and oligo 54.

FIG. 1 is a diagram of a nanosensor embodiment that includes a reporter conjugate bound to an analyte that is itself bound to a capture moiety on a surface.

FIG. 2 is a diagram of a nanosensor embodiment where the analyte is itself a catalytic molecule able to react with a reporter substrate and change the concentration of an effector.

FIG. 3 is a diagram of a nanosensor embodiment with a reporter molecule attached to a surface.

FIG. 4A is a diagram of a nanosensor embodiment with a nucleic acid as the analyte. FIG. 4B is a diagram of a nanosensor embodiment that includes a secondary attachment molecule.

FIG. 5 is a diagram of a nanosensor embodiment with two separate chambers.

FIG. 6A is a diagram of a nanosensor embodiment where the attachment surface is the surface of beads. 6B is a diagram of an activity switch.

FIG. 7A is a schematic drawing of a nanosensor with an area (pad) for capture moiety attachment that is separate from the CNTs. 7B is a schematic drawing of a polymer stamp forming a micro-fluidic channel, upon which lies a surface for capture moiety attachment.

FIG. 8 shows plots of the source-drain current of a CNT conducting path vs gate voltage recorded in air and nitrogen. The bias voltage is 1 V.

FIG. 9 shows plots of the source-drain current of a CNT conducting path vs liquid gate voltages, recorded in buffer equilibrated with nitrogen (A) or with air (B) at one atmosphere pressure, as function of time following reduction of the CNTs. The bias voltage is 0.05 V.

FIG. 10 shows plots of the source-drain current of a CNT conducting path vs liquid gate voltage before and after a pH jump. The bias voltage is 0.05 V.

FIG. 11 is a diagram of hybridization between a surface attached oligonucleotide probe, a nucleic acid analyte, and a different oligonucleotide probe attached to laccase.

### DETAILED DESCRIPTION OF THE INVENTION

The present invention provides a nanosensor for the detection of analytes. Typically analytes of the invention are biomolecules. In the present invention, CNTs are used to detect the presence of an analyte by responding to a change in the concentration of a small effector molecule. The CNTs of the nanosensor are provided with a baseline conductance which will fluctuate relative to a baseline conductance with changes in concentration of the effector molecule.

The present invention provides nanosensors for detecting analytes and methods for their use. The main elements of the nanosensor of the invention are:

An electrical conducting path between at least two electrodes comprised of at least one semiconducting CNT where the CNT maintains a baseline conductance;

An effector molecule in association with the CNT where alterations in the concentration of the effector will alter the conductance of the CNT;

A reporter, typically a catalyst, that acts on a substrate to alter the concentration of the effector;

A capture moiety, attached to a surface where the capture moiety binds the analyte.

Several advantages of this detection system are that 1) the analyte or capture moiety—analyte complex itself does not have to directly change the conductance of the CNT; 2) the capture moiety does not need to be attached to or be in close proximity to the CNT; and 3) the signal is greatly amplified through the reporter molecule.

The present invention also provides methods for detecting an analyte indirectly by first binding the analyte to a capture moiety that is attached to a surface (typically of a support), binding a reporter conjugate to the bound analyte, adding a reporter substrate causing an alteration of an effector concentration to occur, and then measuring the change in conductance of at least one semiconducting CNT in a conductive path that is in contact with a solution containing the effector.

Another embodied method involves binding an analyte that is catalytically active to a capture moiety, adding a reporter substrate causing an alteration of an effector concentration, and then measuring-the change in conductance of at least one semiconducting CNT in a conductive path that is in contact with a solution containing the effector. An additional method for detecting an analyte indirectly is to allow an analyte to react with a surface-attached reporter molecule, causing an alteration of an effector concentration to occur, and then measuring the change in conductance of at least one CNT in a conductive path that is in contact with a solution containing the effector.

Highly sensitive nanoscale detection of biomolecules has utility in bioterrorism, biomedical, environmental, food safety, research, and other applications. Use of the present system wherein detection by the CNT is of a change of effector concentration in solution increases the diversity of biomolecules that may be assayed and the sensitivity of detection. Samples may be screened to detect a target biomolecule that would indicate the presence of a bioterrorism agent, a disease agent, a genetic disorder, an environmental contaminant, a food pathogen, a desired product, and other such components.

The following definitions and abbreviations are to be used for the interpretation of the claims and the specification.

“CNT” means carbon nanotube.

“ABTS⁻²” refers to 2,2′Azino-di-(3-ethylbenzthiazoline-sulfonate).

The term “nanotube” refers to a single-walled hollow cylinder having a diameter on the nanometer scale and a length of several micrometers, where the ratio of the length to the diameter, i.e., the aspect ratio, is at least 5. In general, the aspect ratio is between 100 and 100,000.

By “carbon-based nanotube” or “carbon nanotube” herein is meant a single-walled hollow cylinder composed primarily of carbon atoms.

The term “baseline conductance” refers to the conductance of a carbon nanotube comprised within a nanosensor of the invention, measured prior to the addition of the sample or at the earliest time following the addition of a solution potentially containing the analyte for detection. The baseline conductance provides a measurement that can be compared to the conductance measurement made when the analyte is being detected.

The term “heterogeneous” as used in conjunction with the nanosenor and methods of the invention refers to a sensor or method that makes use of reagent bound to a surface, typically a support. “Heterogeneous” is used antithetically to “homogeneous” where reagents are not bound to a surface. The term “heterogeneous catalysis” refers to catalysis in which the catalytic moiety is attached to a surface either directly or indirectly.

The term “analyte” or “target analyte” means the substance that is the object of detection by the nanosensor. Analytes may be a variety of materials and substances but are typically biomolecules and the product of biological reactions and events. A “catalytic analyte” for example is an analyte that has a catalytic function that has the potential of altering the concentration of an effector by acting on a reporter substate. Catalytic analytes are often enzymes.

The term “target biomolecule” refers to a substance to be detected in a biological sample, or sample potentially containing biological material. The target biomolecule is an analyte that is part of a sample.

The term “capture moiety” refers to a molecule that can interact with and trap a target analyte. When a capture moiety is attached to a surface, the analyte bound to the capture moiety will be attached to the surface as well such that it will not be removed during a wash step.

The term “reporter” will mean a substance capable of reacting with a substrate to alter the concentration of an effector molecule in an environment. The reporter may be chemically or catalytically based. Typical reporters of the invention are enzymes.

The term “reporter conjugate” refers to a molecule with dual functionality. The reporter conjugate is generally comprised of a reporter moiety (generally an enzyme or substance having catalytic activity) and an “analyte receptor”. For the purposes of the present invention the “analyte receptor” will refer to that portion of the reporter conjugate that has specific affinity for the analyte.

The term “reporter substrate” refers to a substrate of the reporter molecule (e.g. enzyme). The reporter substrate is involved in the reporter function which results in the effector molecule being produced or consumed. Typical reporter substrates are enzyme substrates.

The term “effector” or “effector molecule” refers to a small molecule that has the ability to alter the conductance of a semiconducting CNT. Thus, changes in the concentration of the effector can be detected by monitoring changes in the conductance of the CNT.

The term “support” refers to any material comprised within the nanosensor that will serve as a support for the various elements of the sensor, including, but not limited to the CNTs the capture moiety, the reporter conjugate and the analyte. Supports may take a variety of shapes and are composed of a variety of type of materials including polymers, matrices and gels.

The term “surface” refers to the exterior portion of any material. In the context of the present invention a surface will often be located at the solid-liquid interface.

The term “pad” refers to a surface on a support that is a localized area for attachment of the capture moiety. The pad and the rest of the support may each be treated differently so that the pad is prepared for attachment of the capture moiety, and the capture moiety does not attach to the rest of the support.

The term “stamp” refers to a separate support from that on which the carbon nanotubes are placed. The capture moiety may be attached to the stamp surface, or to a portion of the stamp surface.

The term “attach” refers to the affixing to, specifically the affixing of a molecule to a surface such that the attached molecule is not removed from the surface under conditions of the detection process of the invention. Attachment may be directly between the molecule and the surface, or it may be indirectly through an attachment group or secondary attachment molecule.

As used here in the term “source electrode” will mean one of the three terminals of a field effect transistor from which the majority carrier flows into the transistor.

As used herein the term “drain electrode” will mean one of the three terminals of a field effect transistor through which the majority carrier exits the transistor.

As used herein the term “gate electrode” will mean one of the three terminals of a field effect transistor which by means of an electric field controls the flow of charge carriers in the transistor, thereby controlling the output current.

The term “charge carrier” refers to any molecule or other discrete entity that has the ability to receive or donate electrons and carry a charge.

The term “bound” refers to an interaction between two molecules to form a complex. The binding of a first molecule, which is attached to a surface, to a second molecule in solution captures the second molecule so that it is no longer free in solution.

The term “complex” refers to a group of two or more molecules that are bound to each other. Each molecule need not be bound to all other molecules in the complex, but each molecule is bound to at least one of the other molecules such that the group of molecules is held together. Accordingly a “capture-analyte-reporter complex” is a complex formed between an analyte bound to a surface by a capture moiety and the elements of the reporter complex, i.e a reporter and an analyte receptor.

The term “binding partner” refers to one of two molecules that are able to interact with each other such that they form a complex. A “binding pair” consists of two binding partners. Binding pairs particularly suitable in the present invention include, but are not limited to the pair combinations of antigen/epitope, receptor/ligand, binding protein/protein, nucleic acid binding polypeptide/nucleic acid, complementary nucleic acid single strands and peptide nucleic acid and complementary nucleic acid.

The term “polypeptide” refers to a chain of amino acids which may be an entire protein or may be a portion thereof. Polypeptides may be natural or synthetic, and may include one or more artificial chemical analogues of a naturally occurring amino acid. For the purposes of this description a peptide is considered to be a type of polypeptide and a polypeptide is a type of protein.

The term “antibody” refers to a protein or glycoprotein encoded by an immunoglobulin gene, or portion thereof. Any portion of a natural antibody which is able to bind to its antigen, called immunoreactive fragment”, is included in the term antibody. The antibody may be monoclonal or polyclonal. Though natural antibodies are tetramers of two heavy and two light chains, a single heavy and light chain may be adequate for antigen binding, as in Fab fragments. The variable regions of the light and heavy chains are generally the antigen recognition regions of an antibody. Thus a single variable light chain and a single variable heavy chain may suffice for antigen binding, and these may be joined together directly or through a peptide linker.

A “oligonucleotide” or “oligo” is a polymer of RNA, DNA, or peptide nucleic acid (PNA). It optionally contains synthetic, non-natural or altered nucleotide bases. The base sequence of an oligonucleotide probe is complementary to the sequence of the portion of the target nucleic acid molecule to which hybridization is desired. An oligonucleotide probe may also be used to bind to a nucleic acid binding protein. In this case it may be double-stranded if interaction with the binding protein requires a double-strand structure. An oligonucleotide may also be covalently linked to a protein.

The term “peptide nucleic acids” refers to a material having nucleotides coupled together by peptide linkers.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein. The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above, except for the temperature of the final two 30 min washes in 0.2×SSC, 0.5% SDS was increased to 60° C. Another preferred set of highly stringent conditions uses two final washes in 0.1×SSC, 0.1% SDS at 65° C. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater is the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8).

Nanosensor

The invention involves a carbon nanotube based nanosensor capable of detecting the presence of an analyte. The main elements of the nanosensor of the invention are:

An electrical conducting path between at least two electrodes comprised of at least one semiconducting CNT where the CNT maintains a baseline conductance;

An effector molecule in association with the CNT where alterations in the concentration of the effector will alter the conductance of the CNT;

A reporter, typically a catalyst, that acts on a substrate to alter the concentration of the effector; and

A capture moiety, attached to a surface where the capture moiety binds the analyte. In some variations of the sensing format, and depending on the nature of the analyte to be detected, the analyte may not require immobilization via a capture moiety but will be detected via its interaction with an immobilized reporter.

The present nanosensor relies on heterogeneous elements for analyte reporting.

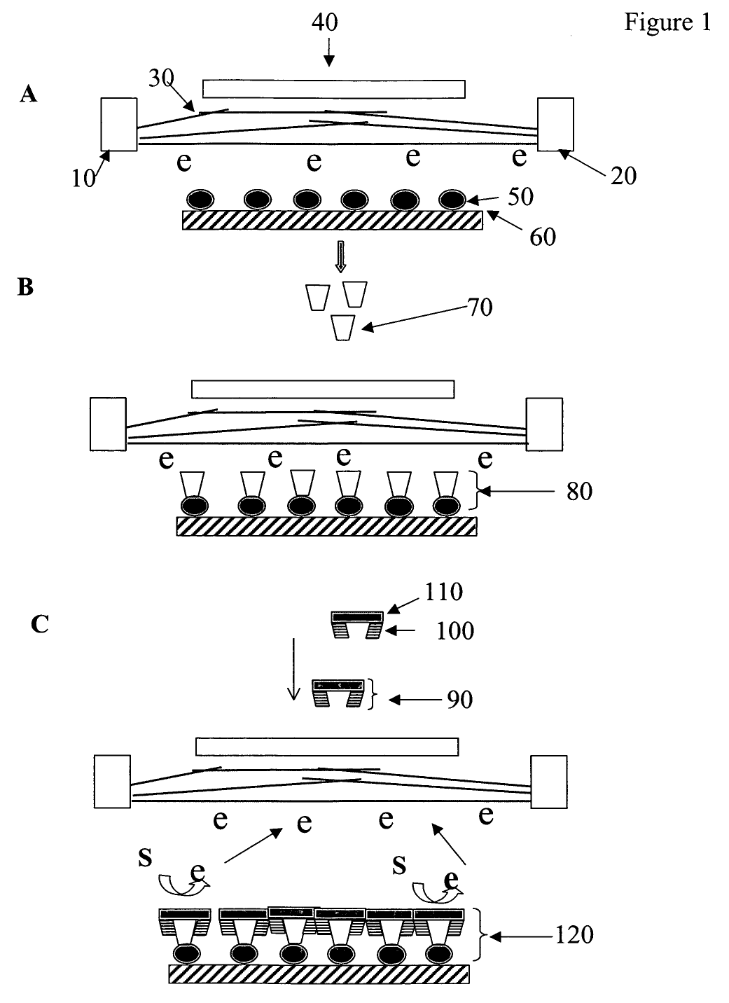

The invention may best be understood by making reference to the figures. The nanosensor of the invention may be used in a variety of formats depending on the nature of the analyte to be detected. For example, one embodiment is shown in FIG. 1. Referring to FIG. 1A, the nanosensor comprises two electrodes (10, 20) connected by an electrically conducting path comprising at least one semiconducting carbon nanotube (CNT)(30). The CNT (30) inherently possesses a baseline conductance. The electrodes (10, 20) may be independently either source or drain. The CNT (30) is in association with an effector molecule (e). The nanosensor additionally may comprise a gate electrode (40) which generates an electric field to gate the conductance of the CNT. The nanosensor additionally comprises a surface (60) having a capture moiety (50) attached thereto. As shown in FIG. 1B, introduction of an analyte (70) to the nanosensor results in the binding of the analyte (70) to the capture moiety (50) to form an analyte-capture complex (80). As illustrated in FIG. 1C, the addition of a reporter conjugate (90) comprising a reporter element (110) and an analyte receptor (100) results in the binding of the reporter conjugate (90) to the analyte-capture complex (80) to form a capture-analyte-reporter complex (120). With the introduction of a reporter substrate (S) the reporter (110) acts on the substrate whereby the concentration of the effector (e) is altered, producing a corresponding alteration in conductance of the CNT (30). Changes in the conductance of the CNT (30) indicate the presence and quantity of the analyte.

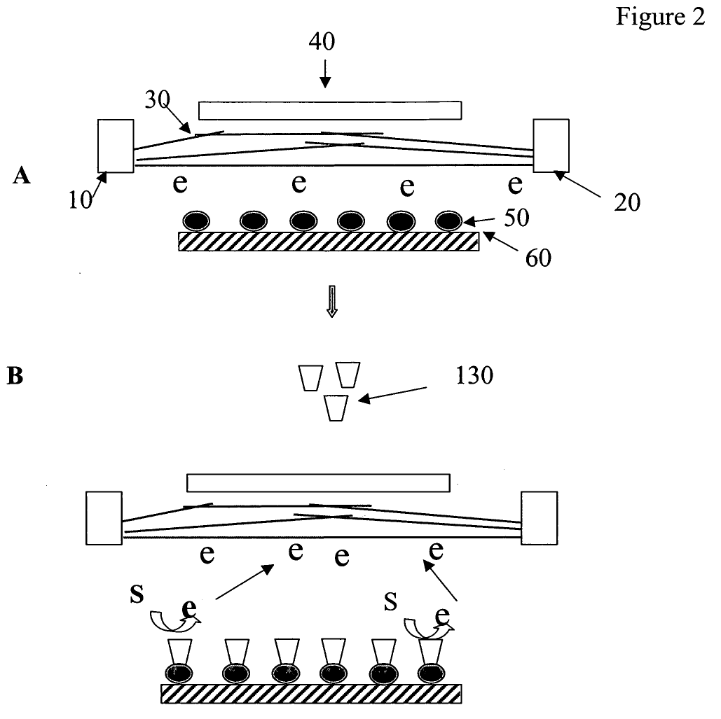

Another embodiment applicable to the detection of a catalytic analyte is shown in FIG. 2. Referring to FIG. 2A, the basic elements of the nanosensor are as illustrated in FIG. 1A. The format of the nanosensor is modified to accommodate the addition of a catalytic analyte (130), typically an enzyme. Because the analyte has catalytic functionality it may act as a reporter. The catalytic analyte (130) binds to the capture moiety (50). A reporter substrate (S) is introduced, where the reporter (110) acts on the substrate whereby the concentration of the effector (e) is altered, producing a corresponding alteration in conductance of the CNT (30). Changes in the conductance of the CNT (30) indicate the presence and quantity of the analyte.

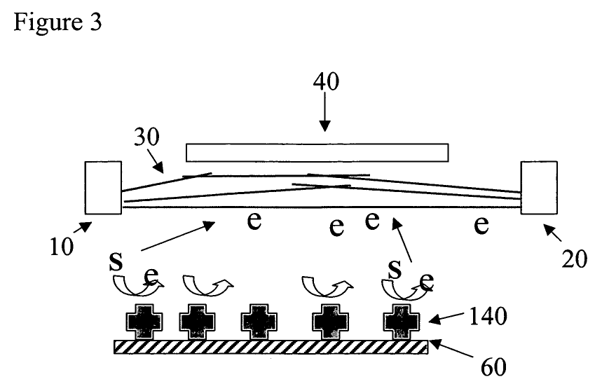

Another embodiment applicable to the detection of an analyte that is a substrate of the reporter. This embodiment is illustrated in FIG. 3. The basic elements of the nanosensor are as illustrated in FIG. 1A. The format of the nanosensor is modified to accommodate a substrate analyte. In this format the analyte is itself a reporter substrate (S), which is acted upon by the reporter (140) producing a change in the concentration of the effector (e).

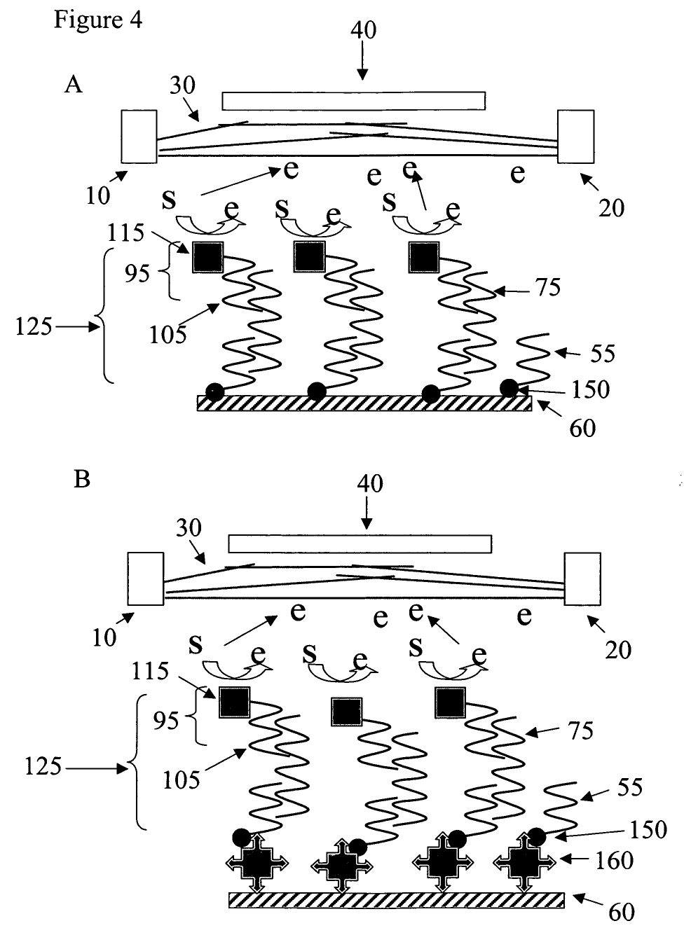

Additional embodiments applicable to the detection of a nucleic acid analyte are shown in FIGS. 4A and 4B. The basic elements of the nanosensor are as illustrated in FIG. 1A. In FIG. 4A the capture moiety (55) is an oligonucleotide that is attached to the surface (60) through an attachment group (150), and has complementarity to a first part of the nucleic acid analyte. The analyte in single-stranded form (75) hybridizes to the capture moiety oligonucleotide (55). The reporter conjugate (95) comprises an analyte receptor (105) that is an oligoucleotide with complementarity to a second part of the nucleic acid analyte, and a reporter element (115). Hybridization occurs between the analyte and the analyte receptor forming a capture-analyte-reporter complex (125). Introduction of a reporter substrate (S) allows it to be acted upon by the reporter, resulting in a change in the concentration of the effector (e) which is detected as a change in conductance in the CNT.

In the embodiment in FIG. 4B all elements are the same as in FIG. 4A with the addition of a secondary attachment molecule (160) that serves to attach the capture moiety (55) to the surface (60).

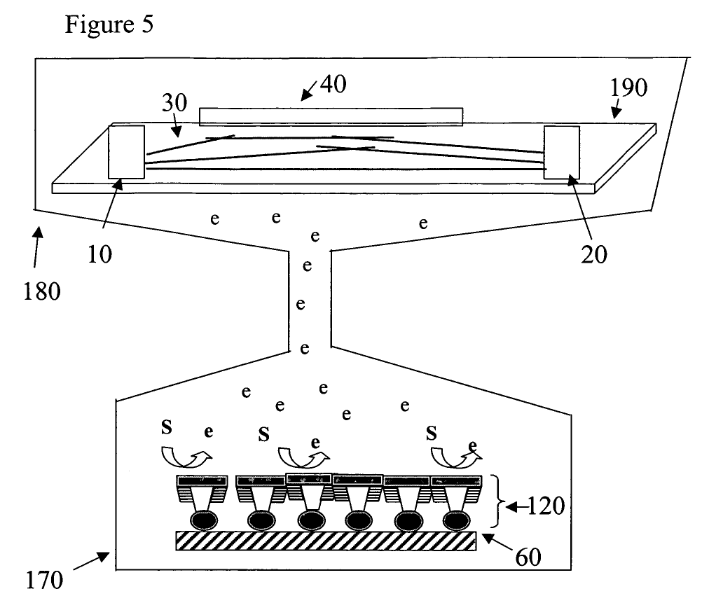

Another embodiment wherein the nanosensor includes two chambers and two surfaces is shown in FIG. 5. Elements of the nanosensor described in FIG. 1 are replicated in FIG. 5. In addition, a first surface (60) with attached capture-analyte-reporter complex (120) is contained within a first chamber (170) that is connected to a second chamber (180) containing a second surface (190) that supports the CNTs (30), and source and drain electrodes (10, 20). The effector flows or diffuses between the two chambers such a change in the effector is detected as a change in conductance in the CNT.

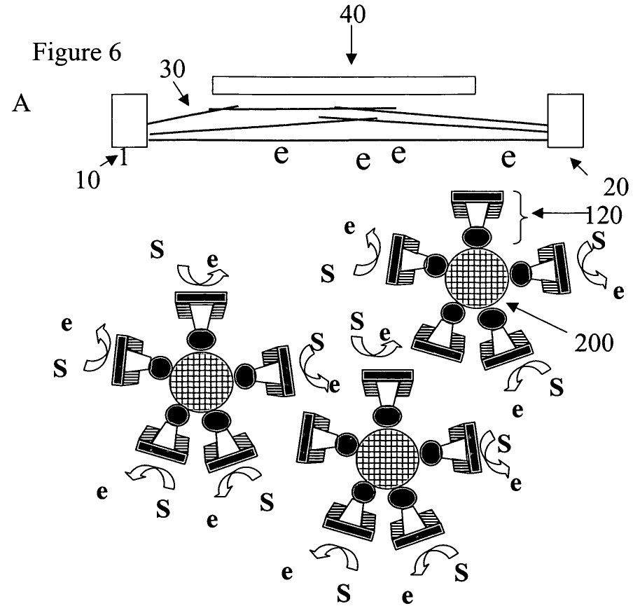

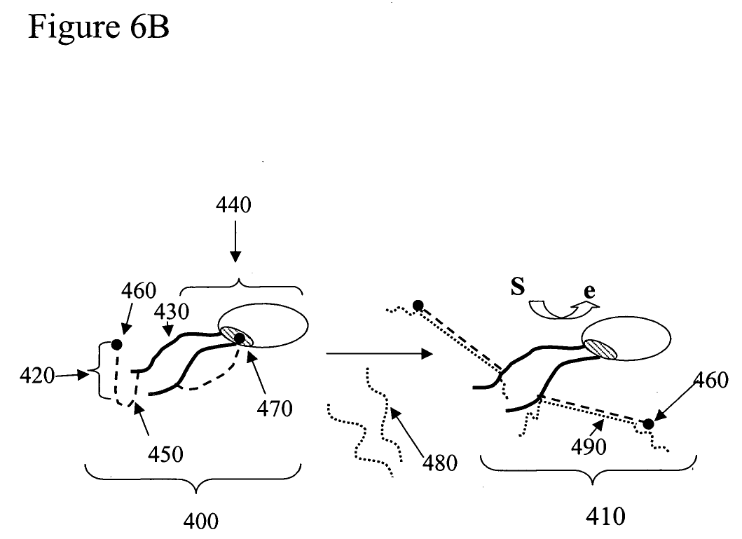

Another embodiment including a surface in the form of beads is shown in FIG. 6A. Elements of the nanosensor described in FIG. 1 are replicated in FIG. 6 with the exception of the surface (60). An alternative type of surface in the form of a bead (200) is the site of attachment of the capture-analyte-reporter complex (120).

In those instances where the reporter is catalytic, the invention provides a format for the nanosensor where the reporter may be activated and “switched on” by the presence of an analyte. Analytes suitable for detection via an activity switch reporter will be those that have the ability to interact with the reporter and “switch on” the reporter. This format employs what is referred to herein as an “activity switch” and allows greater flexibility in the design of the sensor. A specific embodiment of the activity switch is illustrated in FIG. 6B. In this embodiment the reporter is an enzymatic glycoprotein (440), and may exist in either an active (410) or inactive (400) form. One aspect of the glycoprotein (440), is the presence of a point of attachment for an activity switch (420), such as an oligosaccharide chain (430). The activity switch comprises an oligonucleotide (450) anchored via its 5′ end to the glycoprotein (440), and an inhibitor (460) attached to the oligo at the 3′ end. In the inactive form (400) the enzymatic glycoprotein (440) has the inhibitor bound to the active site of the protein (470). When the inhibited reporter comes in contact with an nucleic acid analyte (480) that is complementary to a portion of the anchored oligo (450) the resulting hybridization (490) pulls the inhibitor (460) away from the active site of the glycoprotein (440), thus switching on the enzyme, and conversion of the substrate (S) to an effector (e)

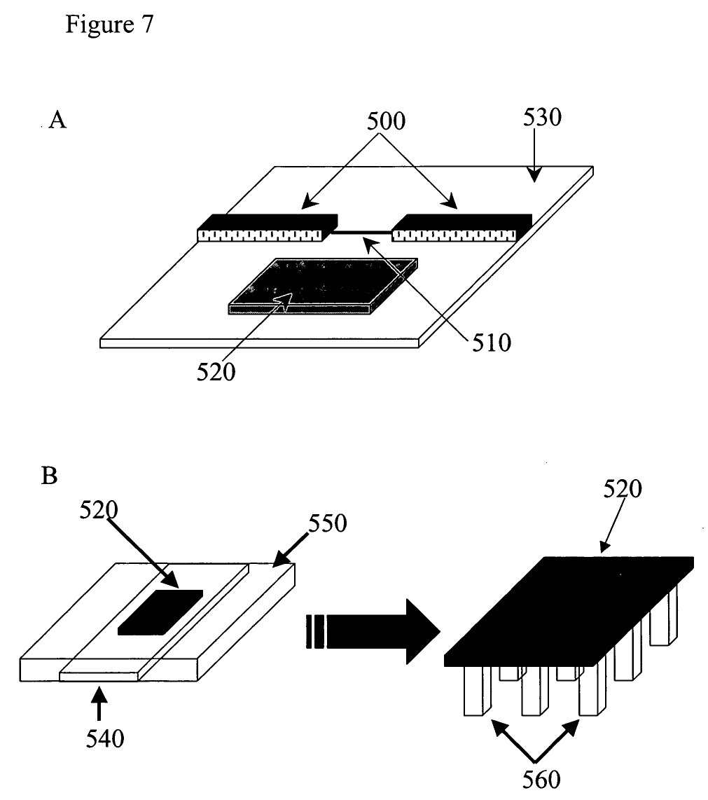

One of the key advantages of the present invention is the ability to detect the presence of an analyte on a surface removed from the carbon nanotube. Several formats of the nanosensor will allow for this type of sensing, one of which is illustrated in FIG. 7. Referring to FIG. 7A, the nanosensor is comprised of a silicon chip (530), supporting a pair of electrodes, (500), the electrodes being connected by an electrically conducting path comprising at least one semiconducting CNT (510). The silicon chip may additionally support a “pad” or functionalized surface which is proximal to, but separated from the CNT (520). The pad may comprise a polymer or metallic surface that is itself a capture moiety or acts in conjunction with one, for example, gold for trapping thiolated oligonucleotides or nickel for trapping magnetic beads.

FIG. 7B shows a schematic drawing of a polymer stamp (550) that may incorporate a pad where the stamp has a micro-fluidic channel (540). Within the channel is a surface for capture moiety attachment (520) that has post-like features (560) to increase the surface area.

One of skill in the art will recognize that features of different embodiments as diagrammed in FIGS. 1-7 may be combined, such as the analyte of FIG. 2 being used in a device with separate chambers as in FIG. 5, and the pad (520) shown in FIG. 7A binding the beads of FIG. 6A. For example, in one particularly useful alternate embodiment, the analyte receptor (an oligo for example) and the reporter (such as an enzyme) may be each independently attached to a bead. Specifically a streptavidin coated bead could bind a biotinylated enzyme (reporter) as well as a biotinylated oligo (analyte receptor). The ratio of reporter enzyme to oligo receptor on the bead could be designed so that the reporter enzyme was in excess to the oligo. Thus, upon the binding (hybridization) of an immobilized analtye (a DNA analyte hybridized to a capture moiety oligonucleotide that is attached to a substrate for example) with the receptor oligo linked to the bead, the presence of the anlayte would be detected by a signal, amplified by the increased concentration of reporter enzyme on the bead. In this fashion many reporter molecules could be associated with each analyte. Additionally, in situations where the bead was magnetic, the magnetic properties could be used to concentrate or purify the analyte.

Supported Carbon Nanotubes

The nanosensor of the invention comprises at least one semiconducting CNT comprised within an electrical conducting path.

CNTs have diameters on the nanometer scale and a ratio of the length to the diameter, i.e., the aspect ratio, of at least 5. In general, the aspect ratio is between 100 and 100,000. Carbon nanotubes are single-walled hollow cylinders composed primarily of carbon atoms. CNTs of the nanosensors of the invention may be doped with agents such as metals and may have coatings. Preferred CNTs are free of metals or coating materials.

CNTs may be produced by a variety of methods, known to those skilled in the art, and are additionally commercially available. Methods of CNT synthesis include laser vaporization of graphite (A. Thess et al. Science 273, 483 (1996)), arc discharge (C. Journet et al., Nature 388, 756 (1997)) and HiPco (high pressure carbon monoxide) process (P. Nikolaev et al. Chem. Phys. Lett. 313, 91-97 (1999)). Chemical vapor deposition (CVD) can also be used for producing carbon nanotubes (J. Kong et al. Chem. Phys. Lett. 292, 567-574 (1998); J. Kong et al. Nature 395, 878-879 (1998); A. Cassell et al. J. Phys. Chem. 103, 6484-6492 (1999); H. Dai et al. J. Phys. Chem. 103, 11246-11255 (1999)).

Additionally CNTs may be grown via catalytic processes both in solution and on solid substrates (Yan Li, et al., Chem. Mater.; 2001; 13(3); 1008-1014); (N. Franklin and H. Dai Adv. Mater. 12, 890 (2000); A. Cassell et al. J. Am. Chem. Soc. 121, 7975-7976 (1999)).

One or more CNTs may be present in the nanosensor as part of an electrically conducting path. The CNTs may take any conformation however single-walled CNTs are preferred. At least one of the CNTs between the source and drain electrodes must be semiconducting to provide an electrically conducting path that can be controlled by a gating electrode. Multiple CNTs of varying chirality may be joined to provide the electrically conducting path.

The CNTs may be suspended between the source and drain electrodes of the nanosensor, or supported on a surface. A gating electrode in the nanosensor generates an electric field to change the CNT conductance such that the sensitivity of the CNTs to the presence of the effector can be optimized. The gate is an electrode separated from the CNT by a dielectric material and polarized relative to the drain electrode. The gate may be a back gate, a top gate or a split gate for operation in air. An electrode that contacts a solution in the CNT chamber may be used for operation as a liquid gate.

Since the concentration of the effector provides the signal for detection by the CNT, there is no requirement for close proximity between the CNT and the capture moiety or reporter. This feature allows the CNT to be in any location accessible either by diffusion or flow, including such transport processes as pumping and injecting, of the effector. For example, the CNT may be on the same surface as the capture moiety or on a surface different from that of the capture moiety as long as the solution containing the effector may contact both surfaces at the same time or sequentially.

The surface of the CNT may be functionalized or coated to enhance or increase the specificity of the detection of the effector concentration. Coatings such as PEG, PEI, PFE, polylysine, polyglutamic acid, and polystyrene sulfonic acid may be added to control non-specific binding or the binding of charged species.

The exact structure of the nanosensor is not specified by the nanosensor of the invention. Any sensor structure may be employed with the components of the invention wherein the CNTs come in contact with a solution in which the concentration of a small molecule effector is altered.

Effector

Small molecules that have the ability to alter the conductance of semiconducting CNTs may be used as effectors in this invention. It is known that oxygen (O₂) and ammonia (NH₃) each are able to significantly change the conductance of CNTs. In addition nitrogen dioxide (NO₂), which is spontaneously formed from nitric oxide and oxygen, can alter conductance of CNTs allowing this small molecule to be an additional effector. Also hydrogen ions (H⁺) may be used as an effector, since a change in their concentration affects the conductance of CNTs. Other small molecules may be identified as being able to alter CNT properties, and as such may also be appropriate effectors for use in this invention. The effector is in solution and must contact the CNTs. Typically effectors are consumed or produced by the interaction of a reporter and a reporter substrate.

Analyte and Capture Moiety

The present invention is designed to detect the presence of an analyte. The analyte may be any substance that is amenable to being captured and immobilized near a surface, however biomoleclues are particularly suitable. Analytes that are targets may be, for example, chemicals and biomolecules. Preferably, biomolecules are analyte targets of the invention. An analyte may be a nucleic acid that is either DNA, PNA (peptide nucleic acid) or any type of RNA, for example ribosomal RNA, messenger RNA, and antisense RNA. An analyte may be a protein, a polypeptide, or a peptide. An analyte may be a virus, a cell such as a prokaryotic cell including a bacterial cell, or a eukaryotic cell such as a plant or an animal cell. An analyte may be a metabolite of a biological organism or a product produced by a biological organism.

The capture moiety binds the analyte when it is present in the test sample. The capture moiety may be any molecule that can bind to an analyte and the two form a binding pair, with each being a binding partner. The binding pair may be two polypeptides, a polypeptide and a nucleic acid molecule, a polypeptide and a chemical, two chemicals, a nucleic acid molecule and a chemical, or any type of combination where the analyte is one member. If an analyte is a protein, an antibody recognizing an epitope of that protein is a binding partner. If an analyte protein naturally binds to another protein such as two proteins in an enzyme complex, these two proteins are binding partners. Another type of protein/protein binding pair is a protein and its receptor. In any of these cases, either the entire protein or only the binding portion of a protein may be used as a binding partner. If an analyte is a DNA molecule, having a sequence that naturally binds to a protein such as a transcription factor, then the transcription factor protein or a binding portion thereof and the target DNA molecule form a binding pair. Examples of well-known binding pairs include hapten/anti-hapten, glutathione-S-transferase/glutathione, 6× histidine Tag/Ni-NTA, streptavidin/biotin, S-protein/S-peptide, cutinase/phosphonate inhibitor, folic acid/folate binding protein, protein A/protein A immunoglobulin and protein G/protein G immunoglobulin.

In addition, complementary single stranded nucleic acid molecules are binding partners. The capture moiety binding partner has a sequence complementary to at least a portion of the sequence of a target nucleic acid. If a target nucleic acid molecule is double stranded, then the capture moiety may have complementary sequence to either strand of the target. The strand that is captured by the capture moiety will usually also be complementary to the analyte receptor oligonucleotide. Prior to detection the double stranded DNA is melted into two free single strands. Capture of a nucleic acid single strand and the steps that follow are carried out below the melting temperature. One skilled in the art will know the length of the nucleic acid strand required to have stable hybridization and the conditions of the assay required to maintain the double strand during detection. In addition triple strand nucleic acid formations may be used as capture moiety, analyte, and analyte receptor. Methods for hybridization are well known in the art, see for example Sambrook supra.

An optional aspect of the present invention is to distinguish a nucleic acid molecule target that is completely complementary to an oligonucleotide probe capture moiety from one that has one or more mismatches. Assay conditions such as salt concentration and temperature may be adjusted to allow hybrids only of completely matching nucleic acids to be stable. Determination of appropriate hybridization stringency conditions to use with a specific polynucleotide probe and target nucleic acid molecule pairs is well known to one skilled in the art.

Capture Moiety Attachment

The invention relies on the presence of a capture moiety to immobilize the analyte on a surface. Generally the capture moiety is affixed on the surface or support. The capture moiety may take a variety of forms. In some instances the capture moiety will bind the analyte which will additionally be bound to a reporter conjugate as described in FIG. 1. Alternatively the capture moiety will bind the analyte, which will itself serve the reporting function as described in FIG. 2. In some instance the reporter will itself will be immobilized on the surface (as described in FIG. 3).

Irrespective of its form, the capture moiety or reporter must be affixed to a surface. That surface may be the surface of the carbon nanotube of the nanosensor but is more typically and preferably the surface of a support. The capture moiety may be on the same support surface as the CNTs or on a separate support surface from the CNTs.

Nanosensor Surfaces and Supports

The nanosensor may include a first and a second support. The first support, when included, provides a surface for placement of the CNTs and source and drain electrodes which form an electrically conducting path. The first surface may be comprised of any non-conductive material. Examples include, but are not limited to, silicon, polysilicon, silicon dioxide, silicon nitride, polymeric materials, glass, agarose, nitrocellulose, nylon, and insulating materials. Particularly useful are silica chips. Typically silica chips have a thin layer of natural oxide, which has very low electrical conductivity and is an insulator. For better insulation of the surface from the underlying silica, a thicker oxide layer that is typically about 500-600 nm may be added, by a method such as with a thermal treatment in air, for example. This provides additional insulation from the underlying silica.

The function of the second surface or support is for the attachment of the capture moiety. The surface to which the capture moiety is attached may be located adjacent to the CNTs, not adjacent but within the same chamber as the CNTs, or in a separate chamber from the CNTs, as long as the solution with altered effector concentration can contact the CNTs. In addition, the second support may be the same as, or the same as a portion of, the first support. Some materials which may be used in a second support include silicon, silicon dioxide, silicon nitride, polysilicon, polymeric materials, glass, agarose, nitrocellulose, nylon, ferromagnetic materials, carbon, metals such as gold and nickel and insulating materials as well as semiconducting materials and functional polymers.

Preferably the capture moiety is on a surface that is within about 100 μm from the sensing CNTs. Where the surface is a support, the support may be of any shape, including for example a stamp, a sheet or film, a sphere such as a bead, and a tube. A bead may be for example a magnetic bead, a hydrogel bead, a solid bead, a silica coated bead, or other type of bead. Particularly suitable is a surface area of about 10⁴ μm², one example of which is diagrammed in FIG. 7A. The capture moiety surface area, as exemplified in FIG. 7A, may be a support such as a pad or a stamp. An example of a pad is an area of gold to which a capture moiety oligonucleotide may be attached using a thiol group. An example of a stamp is deposition of silane in the area for oligonucleotide capture moiety attachment. In designing the stamp it may be useful to pretreated the surface with silicon nitride except in the stamp area to block silane binding, and thus block capture moiety attachment.

Particularly useful is a support surface that is different from that on which the carbon nanotubes are placed. This separate surface can be processed independently of the CNT support surface and placed onto the CNT support surface as a stamp. This nanosensor structure allows the use of different stamps with the same CNT support, thus reducing the setup time and total cost. The stamp may be made using various types of polymers including resins, epoxies, and flexible polymers like polydimethylsiloxane (PDMS), using standard molding processes. Molds may be of any material where features on the mold can be made at a micron scale such as metallic, glass, quartz, silicon and polymer molds. The stamp may have different types of molded surfaces—e.g. flat or channeled. In a particularly useful design, a separate functional polymer stamp has a micro-fluidic channel to which the capture moiety is attached (FIG. 7B). The channel is placed over the CNTs and pressed to the CNT support surface. Fluids containing analytes and detection components of the nanosensor are passed through the channel, making contact with the capture moieties and allowing CNT sensing of changes in concentration of the effector. The surface bearing capture moieties may include post-like features (FIG. 7B) in order to increase the surface available for capture moiety attachment, thus increasing the sensitivity of detection. This type of capture moiety attachment structure may make up a portion of the stamp, with the post-like features extending into the micro-fluidic channel. Alternatively, polymer or silica-based beads with attached capture moieties may be deposited on top of the stamp following capture of the analyte and binding of the reporter conjugate.

An attachment group may be added to a capture moiety to use in attaching it to a surface. For example, a capture moiety molecule may be attached to a gold surface using a thiol group. The capture moiety molecule may be linked to a silica surface using a silane containing reactive groups.

Optionally, the capture moiety may be attached to a surface through non-covalent interaction with a secondary attachment molecule. For example, streptavidin may be attached to the surface and the capture moiety may be derivatized with biotin so that it will be attached to the surface through the interaction between biotin and streptavidin. Additional examples of such secondary attachment systems include glutathione-S-transferase/glutathione, 6× histidine Tag/Ni-NTA, S-protein/S-peptide, cutinase/phosphonate inhibitor, antigen/antibody, hapten/anti-hapten, folic acid/folate binding protein, and protein A or G/immunoglobulins. The fabrication of areas for capture moiety attachment can be achieved using methods such as lithography, micro-contact printing and ink-jet printing of molecules tethered to functional groups, which can anchor capture moiety molecules.

Optionally the surface may be coated with a material to facilitate attachment of the capture moiety. For example, the coating may be a polymer such as poly(ethylene imine) that provides amine groups to facilitate binding of proteins or polynucleotides. The surface may be silanated using a silanation reagent. One skilled in the art will be familiar with different silanation reagents that may be used to add different reacting groups such as amines, carboxylic acids, thiols, and aldehyde groups onto surfaces to act as attachment sites. For example aldehyde groups may be added to surfaces for attachment of oligonucleotides containing hydrazide or amine linkers. Examples of chemistries that may be used for attachment with silane reactive groups include hydrazine-aldehyde chemistry and succinimidyl-6-hydrazinonicotinate acetone hydrazone (SANH) chemistry.

Following capture moiety attachment, a surface may be treated in order to reduce non-specific binding of molecules. This treatment or passivation of the surface may include not only the surface for capture moiety attachment, but also the surface of the CNT support (where separate), especially between the electrodes. For example, when nucleic acid analytes and probes are to be used in a nanosensor, reduction of non-specific binding may be achieved by (1): coating the surface with blocking reagents (e.g. polyethylene glycol) or (2) blocking solutions (e.g. casein), both of which prevent non-specific binding of nucleic acids.

Reporter Molecules, Reporter Coniugates and Reporter Substrates

The reporter of the invention is responsive to the presence of an analyte and interacts with a reporter substrate. The reporter molecule may be any molecule that alters the concentration of an effector in solution in the presence of a reporter substrate. The effector is either produced or consumed as a result of that interaction. Enzymes may be used as reporter molecules and reporter substrates may be enzyme substrates. The enzyme reporter catalyzes a reaction involving the reporter substrate that results in changes in the effector concentration.

Oxidases such as glucose oxidase, laccase, alphahydroxy acid oxidase, aldehyde oxidase, L-amino acid oxidase, ascorbate oxidase, cholesterol oxidase, bilirubin oxidase, and xanthine oxidase can be used as reporter molecules with oxidizable reporter substrates. For example, glucose oxidase causes the reporter substrate glucose to be oxidized by oxygen to produce hydrogen peroxide and gluconolactone. This reaction decreases the concentration of the effector molecule, oxygen, which, in turn alters the baseline conductance of the associated CNTs. Laccase reduces oxygen to water in the presence of an oxidizable reporter substrate such as phenols, thereby decreasing the oxygen concentration. Ammonia production is accomplished for example using as the reporter molecule/reporter substrate combination: glutaminase/glutamine, asparaginase/asparagine, and urease/urea. Other examples of ammonia producing reporter molecules are amidase, formamidase, arginase, and ammonia lyases. Decreases in concentration of an ammonia effector can be accomplished using for example reporter molecules glutamine synthase, and asparagine synthase with glutamatic acid and aspartic acid as analytes, respectively.

The effector molecule nitrogen dioxide may be produced using nitric oxide synthase, where the nitric oxide produced will be converted to nitrogen dioxide in the presence of oxygen. When H⁺ is the effector, enzymes such as urease and various types of esterases, nucleases, and phosphatases act on their substrates, which are used as reporter substrates, and cause the H⁺ concentration to be decreased (pH to rise).

More than one reporter molecule may be used in a cascade of reactions that alter the concentration of an effector in the presence of an analyte. Examples are the combination of glucose oxidase and catalase that result in the oxidation of glucose to gluconolactone and the consumption of O₂ but without accumulation of H₂O₂. Similarly D-amino acid oxidase and monoamine oxidases each produce H₂O₂ and NH₃. The presence of catalase would assure the disproportionation of H₂O₂ to O₂ and water, resulting in a both consumption of O₂ and production of NH₃. These two effects reinforce each other to switch on the carbon nanotube conductance at more positive gate voltages.

In the reporter conjugate the reporter molecule is linked to an analyte receptor that binds to the analyte. The analyte receptor and analyte together form a binding pair, with each being a binding partner or a single member of a binding pair. The binding pair may be two polypeptides, a polypeptide and a nucleic acid molecule, a polypeptide and a chemical, two chemicals, a nucleic acid molecule and a chemical, or any type of combination where the analyte is one member. If an analyte is a protein, an antibody recognizing an epitope of that protein is a member of a binding pair. If an analyte protein naturally binds to another protein such as two proteins in an enzyme complex, these two proteins are a binding pair. Another type of protein/protein binding pair is a protein and its receptor. In any of these cases either the entire protein or only the binding portion of a protein may be used as a binding partner. If an analyte is a DNA molecule having a sequence that naturally binds to a protein such as a trancription factor, the transcription factor protein or the binding portion thereof and target DNA molecule form a binding pair. Examples of well known binding pairs include hapten/anti-hapten, glutathione-S-transferase/glutathione, 6× histidine Tag/Ni-NTA, streptavidin/biotin, S-protein/S-peptide, cutinase/phosphonate inhibitor, folic acid/folate binding protein, protein A/protein A immunoglobulin and protein G/protein G immunoglobulin.

In addition, complementary single strand nucleic acid molecules are members of binding pairs. The analyte receptor binding partner has a sequence complementary to at least a portion of the sequence of a target nucleic acid molecule. If a target nucleic acid molecule is double stranded, then the analyte receptor may have complementary sequence to either strand of the target. The strand that is captured by the analyte receptor will usually also be complementary to the capture moiety polynucleotide. Prior to detection, the double stranded DNA is melted into two free single strands. Capture of a nucleic acid single strand and the steps that follow are carried out below the melting temperature. One skilled in the art will know the length of analyte receptor required to have stable hybridization and the conditions of the assay required to maintain the double strand during detection.

Linking of a reporter molecule with an analyte receptor is accomplished such that activity of the reporter molecule with respect to effector production or consumption is maintained. Processes for protein-protein or protein-nucleic acid linking are well known to one skilled in the art (G. T. Hermanson (1996)—Bioconjugate Techniques, Academic Press, New York).

Where the reporter is an enzyme the use of a reporter molecule or a reporter conjugate results in greatly increased sensitivity of analyte detection due to amplification of the signal by the catalytic turnover of the reporter molecule. Many more effector molecules are produced or consumed in the catalytic reaction mediated by the reporter molecule than the number of analyte molecules captured from the test sample. This amplification process allows the assay of microliter size samples containing limited analyte molecules and makes the system amenable to small scale screening applications.

If the analyte is itself an enzyme that catalyzes a reaction involving the reporter substrate that results in a change in effector concentration, then no additional reporter conjugate is required in the nanosensor (as in FIG. 2).

Reporter Molecule with Activity Switch

In one embodiment the reporter molecule may be modified to include an activity switch that can regulate the enzymatic activity of the reporter molecule. In this embodiment, the reporter molecule with the activity switch is attached to a surface, and no separate capture moiety is included.

In the present invention the activity switch has two components: an inhibitor that binds to the active site or an allosteric site of the reporter molecule (enzyme) thereby blocking its activity, and an analyte receptor that binds to the target analyte. An inhibitor is particularly useful when the dissociation constant of the inhibitor for its binding site on the enzyme is substantially lower than that of the substrate for its binding site, which may or may not be at the same site. Where the reporter can bind either the substrate or the inhibitor, but not both at the same time, then this difference in dissociation constant assures that the inhibitor is able to prevent the substrate from binding (competitive inhibition).

The activity switch may be attached to the reporter molecule directly, or if the reporter molecule has oligosaccharide chains, the activity switch may be attached to these chains. For example, the enzymatic glycoproteins glucose oxidase and laccase have oligosaccharide chains which are locations for activity switch attachment.

In the present embodiment the analyte receptor may be any molecule which can bind to the target analyte and which allows the inhibitor to access the active site or allosteric site in the free state but does not allow access upon binding to the target analyte. The analyte receptor may be, for example, a protein or a nucleic acid. Preferred is a single stranded oligonucleotide probe, attached via the 5′ end to the reporter molecule and linked at the 3′ end to an inhibitor of the enzyme activity. It is understood that the attachments at the 5′ and 3′ ends can be switched without impact on the function. The oligonucleotide, which is highly flexible in its single stranded form, is able to bend such that the inhibitor binds to the active site or allosteric site, blocking the action of the enzyme on its reporter substrate. Upon hybridization of the complementary strand of the analyte DNA (or RNA) to the enzyme-bound oligonucleotide probe, the double stranded DNA (or DNA/RNA hybrid) is now much more rigid than the single strand, with a persistence length some 60-fold greater than that of the single stranded probe oligonucleotide. The inhibitor can then no longer bind to the active site or allosteric site of the enzyme, which is turned on. The active enzyme is now able to process a reporter substrate and change the concentration of the effector. One skilled in the art will know the length of analyte receptor required to have stable hybridization and the conditions of the assay required to maintain the double strand during detection. It is particularly useful for hybridization of the oligonucleotide analyte receptor to the analyte nucleic acid to drive the dissociation of the inhibitor from its binding site. This occurs when the decrease in free energy associated with hybridization of the analyte receptor to the analyte exceeds that associated with the binding of the inhibitor to the enzyme.

One key utility of the activity switch is that its use eliminates the need for separation of components of the nanosensor. Where the activity switch is being employed, the assay may take place in the presence of the enzyme substrate and washing the nanosensor to remove excess enzyme would not be necessary. This is the case because the enzyme reporter is only active and able to receive substrate when the analtye has been bound by the analyte receptor.

A preferred embodiment of the activity switch is an oligonucleotide analyte receptor attached at one end to a laccase inhibitor and at the other end to a laccase enzyme. Preferred laccase inhibitors are tetrachloro-o-quinol, tetrafluoro-o-quinol, 3,4-dihydroxy-2,6-dichloro-benzaldehyde, and substituted hydrazone of 3,4-dihydroxy-2,6-dichloro-benzaldehyde. Any methods for attaching compounds to DNA, and DNA to proteins may be used. Preferred attachment of the inhibitor to DNA is through a maleimide group on maleimide-dPEG₄-NS ester coupled to 3,4-dihydroxy-2,6-dichloro-benzaldehyde through hydrazine derivatization. Preferred attachment of DNA to laccase is by aldehyde-hydrazide attachment chemistry.

Removing Unbound Target Analyte and Unbound Reporter Conjugate

Unbound molecules are removed at specified stages during the analyte detection processes shown in FIGS. 1 and 2. Unbound analyte that did not bind to surface attached capture moiety molecules is removed. Likewise, any reporter conjugate that is not associated with bound analyte is removed. Removing these unbound molecules may be achieved by washing with an appropriate buffer, as would be known to one skilled in the art.

Direct Analyte Detection

An analyte may directly change the concentration of the effector without adding a reporter molecule or reporter substrate. For example, aldehydes, ketones, alkynes and acid chlorides react with ammonia. These types of analytes would themselves reduce the concentration of ammonia when added to a solution containing ammonia. A solution may be pre-loaded with ammonia in order to detect the presence of this type of analyte. Dienes undergo an autooxidative and photooxidative reaction in the presence of oxygen. Thus a diene analyte incubated in a solution containing oxygen would reduce the concentration of the effector molecule oxygen under illumination.

Samples

Samples that may be assayed for the presence of an analyte using nanosensors and methods of the present invention include biological samples as well as non-biological samples. For example, a sample may be from a cell, tissue or fluid from a biological source including a human, an animal, a plant, fungus, bacteria, virus, etc. The source of a sample is not limited and may be from an environmental source, from food or feed, produced in a laboratory, or other source.

Method for Analyte Detection

In methods for analyte detection shown in FIGS. 1 and 2, components of the nanosensor are added in stages or steps. The sample is added such that it is in contact with the capture moiety allowing the analyte to bind to the capture moiety. After binding goes to completion, analytes that did not bind to the surface-attached capture moiety molecules are removed. Removing these unbound molecules may be achieved by washing with an appropriate buffer, as would be known to one skilled in the art. If a reporter conjugate is included in the embodiment followed, a solution of the reporter conjugate is then added such that it is in contact with the capture moiety-analyte complex. Following binding, any reporter conjugates that are not associated with the capture moiety-bound analyte are removed, again by washing. The analyte solution may also be placed in contact with the reporter conjugate first and with the capture moiety either secondarily or simultaneously, after which unbound species are removed by washing. The reporter substrate is added such that it is in contact with the reporter molecule or a catalytically active analyte and the effector concentration is altered as a result.

In the method for analyte detection shown in FIG. 3, the sample is added such that it is in contact with the reporter molecule and the effector concentration is altered as a result of interaction between the reporter molecule and the analyte.

In all three methods for analyte detection, the solution with the altered effector concentration may already be in contact with the CNTs if the CNTs and capture moiety or reporter molecule components are in the same chamber. If the CNTs and the capture moiety or reporter molecule are in separate chambers, then the solution with altered effector concentration is brought in contact with the CNT. The solution containing the effector may flow through a channel, tubing, or other conduit to come in contact with the CNT. The conductance of the CNT is then measured and compared to a measure of the CNT conductance that was taken either prior to adding the sample or at the earliest time after adding the sample. Measurement of the CNT conductance is made by applying a dc (direct current) bias voltage between the source and drain electrodes while varying the gate voltage. In addition, the signal to noise ratio may be improved by ac (alternating current) modulation of the bias voltage. Alternatively, the CNT conductance is measured by holding the gate voltage constant and recording the current as a function of time. A gate electrode is preferred but not required.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “μL” means microliter(s), “mL” means milliliter(s), “nm” means nanometer(s), “mm” means millimeter(s), “cm” means centimeter(s), “μm” means micrometer(s), “mM” means millimolar, “M” means molar, “V” means volts, “mV” means millivolts, “Vg” means gate voltage, “Vsd” means source-drain voltage, “Isd” means source-drain current, “p-type” means charge carrier type (e.g. hole), “CVD” means chemical vapor deposition.

### Example 1

### Carbon Nanotube Response to Oxygen in Gas Phase

Nanotube devices, prepared as follows, were purchased from Molecular Nanosystems (Palo Alto, Calif.). Single-walled carbon nanotubes were grown from catalyst pads in a CVD furnace at 900° C. The catalyst pads were patterned on a thermally oxidized surface (500 nm thick) of a (100) silicon wafer. After the growth, less than or equal to 5 nm of Ti, 50 nm of Pd and less than 50 nm of Au layers were deposited sequentially onto the SiO₂/Si surface to form electrical contacts with the carbon nanotubes.

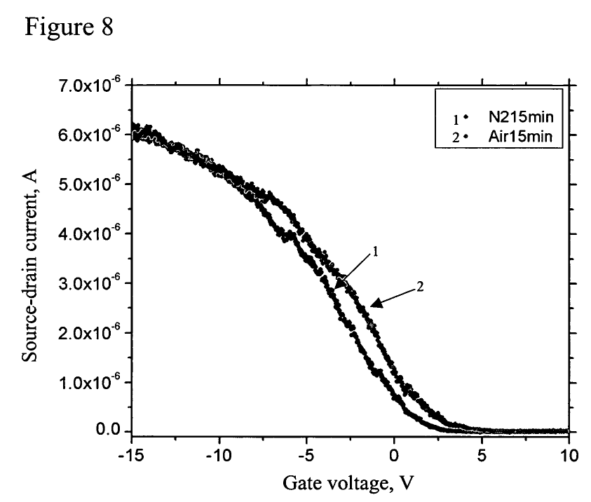

The metallic nanotubes present in the gap (2 micron) were destroyed, by ramping the bias voltage from 0 to 10V while holding the back gate voltage at 0V. This procedure performed in air, enhanced the ON-OFF ratio of the devices to ˜3-4 orders of magnitude. The electronic properties of the remaining semiconducting nanotubes were monitored by applying a dc bias voltage between the source and the drain electrodes while changing the back gate voltage. A flow cell of 4.4 μl volume was mounted and sealed around the carbon nanotube device using an O-ring to allow control of the surrounding atmosphere. The nanotube devices were first characterized in air and then under a nitrogen atmosphere. The plots of the source-drain current vs gate voltage recorded in air and nitrogen are presented in FIG. 8. The plot recorded in nitrogen atmosphere was shifted toward negative gate voltages relative to the plot recorded in air. This shift is explained by the following events. As nitrogen gas is passing through the flow cell placed on top of the nanotube device, the amount of oxygen in the atmosphere and that attached to the nanotube decreases with time. The removal of oxygen from the sidewalls of the nanotube results in an injection of electrons back into the nanotube, where electron-hole recombination takes place spontaneously, thus decreasing the concentration of the free p-type carriers, which makes the device harder to turn ON. The turn ON voltage depends greatly on the environment of the nanotube and also reflects the concentration of the free charge carriers in the nanotube. As soon as the nitrogen gas is replaced with air, the current vs gate voltage plot shifts back to the original position recorded previously indicating that oxygen was reunited with the nanotube.

This experiment, carried out in gas phase, clearly indicated that carbon nanotube based devices are adequately sensitive to oxygen and can be used for oxygen-mediated sensing applications.

### Example 2

### Carbon Nanotube Response to Oxygen in Liquid Environment

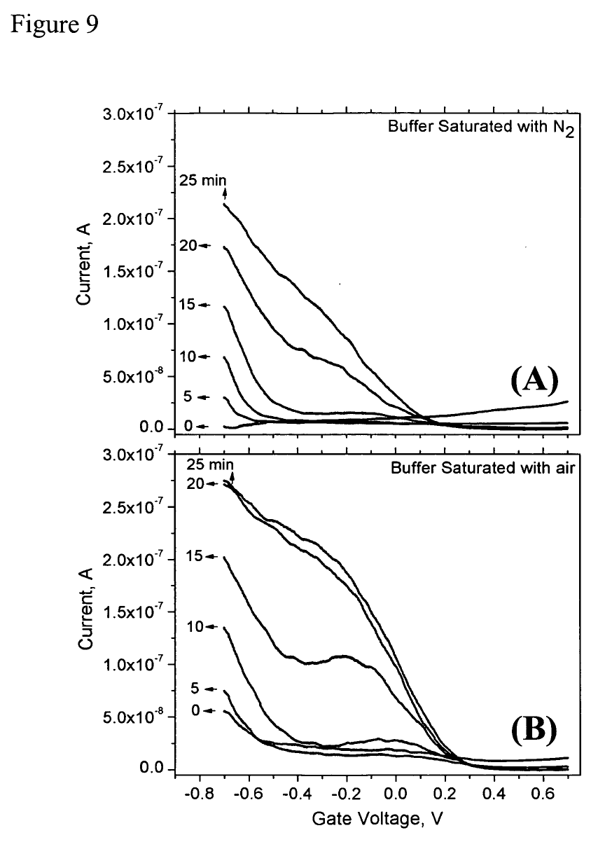

In the previous example, the source-drain current was monitored as a function of the gate voltage applied to the back gate. Here liquid gating was used to control the conductance of the nanotube. To operate in liquid gating mode a third electrode, in addition to the source and the drain electrodes, was submerged in solution that was injected into the flow cell chamber. The source-drain current vs liquid gate voltage characteristics in 50 mM glycine buffer at pH=3 were similar to those recorded in air using the back gate. However, once the solution of a 1 to 1 ratio of the redox couple ferricyanide (K₃Fe(CN)₆)/ferrocyanide (K₄Fe(CN)_{6) (}1 mM total concentration) was added into the cell chamber, a large shift toward more negative gate voltages occurred immediately. This shift is due to the reduction of the carbon nanotube at the redox potential defined by the ferricyanide/ferrocyanide ratio. After equilibration, the cell chamber was washed thoroughly with fresh 50 mM glycine buffer to remove ferro- and ferricyanide molecules. Recovery was monitored over time in the presence and absence of oxygen. The rate of recovery depended greatly on the concentration of oxygen in the buffer solution. FIG. 9 shows the source-drain current as a function of liquid gate voltages recorded 5, 10, 15, 20 and 25 minutes after fresh buffer that had been equilibrated with nitrogen (FIG. 9A) or with air (FIG. 9B) was injected into the cell chamber. In the case of air-equilibrated buffer, the recovery was rapid and the Isd vs gate plot shifted halfway back after 15 minutes. However, in the case of nitrogen-equilibrated buffer the recovery was slowed and the Isd vs gate plot shifted halfway after 20 minutes. The total recovery was also greater in the buffer equilibrated with air than with that equilibrated with nitrogen. The faster recovery can be explained by the oxidation of the nanotube by oxygen molecules present in the air-equilibrated buffer. The oxidation of the carbon nanotube causes an increase in the number of p-type charge carriers and makes the nanotube more p-type, thus producing a shift toward positive gate voltages.

In this example, the sensitivity of the carbon nanotubes to oxygen molecules in a liquid phase environment was demonstrated. Based on this finding, biomolecules such as DNA can be detected by their induced attachment of a reporter molecule that consumes oxygen.

### Example 3

### Carbon Nanotube Response to Hydrogen Ions in Liquid Environment

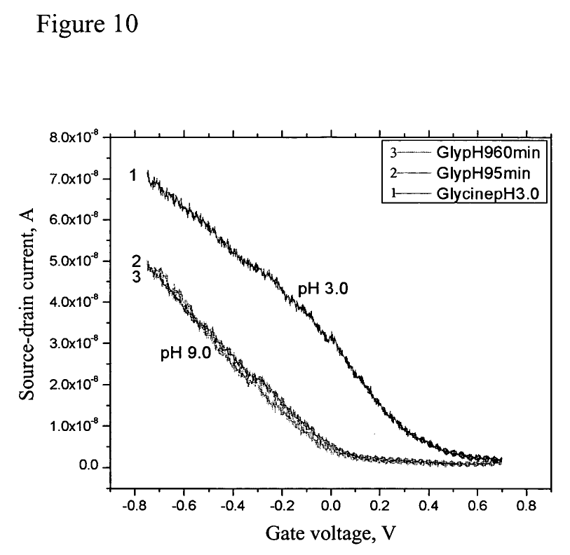

The liquid flow cell mounted on the nanotube device was initially filled with a 50 mM glycine buffer, pH 3.0 and the source-drain current vs liquid gate voltage characteristics were recorded. The Isd vs. Vg plot is shown in FIG. 9 as curve 1. The buffer was then replaced with 50 mM glycine buffer, pH 9.0 over a period of 5 min and the Isd vs.Vg curve was re-measured under the same conditions (FIG. 10, curve 2). A shift of the Isd vs Vg curve toward negative gate voltages occurred. Incubation for an additional 55 min under the same conditions produced little further change in the Isd vs.Vg curve (FIG. 10, curve 3).

The response of the nanotube to the pH of the solution makes it possible to use the nanotube as a pH sensor and a detector for any process that results in a change in pH or hydrogen ion concentration.

### Example 4

### Attachment of Oligonucleotides to Silica Chip Surfaces Using Hydrazide-Aldehyde Chemistry

Silica chips were coated by vapor deposition with triethoxy-silyl-butyraldehyde by incubation in an evacuated chamber for 6 h in the presence of a pool of liquid silane. The chips were rinsed with acetone and isopropanol, and dried under a stream of N₂. The chips were then dried either by heating in a vacuum oven for 1 h at 100° C. or left overnight under dry N₂. They were then stored in a glove box under N₂ until use.

An oligonucleotide, oligo 28 (SEQ ID NO:1), was designed from the human gene encoding low-density lipoprotein receptor (LDLR, oligo beginning with codon 194), and oligo 29 (SEQ ID NO:2) was designed as the oligo 28 complement. Oligonucleotides 28 and 29, each with a 5′-linked hydrazide, were purchased from Integrated DNA Technologies, Inc. (Coralville, Iowa). These 5′-hydrazide-linked single-stranded oligonucleotide probes (10 μM) in 0.5 M NaHPO₄, pH 7.4 were incubated for 1 h on a silane-treated chip, after which the DNA solution was made 25 mM in NaBH₃CN and incubation continued for another 45 min. Incubation was carried out in Petri plates maintained at 100% relative humidity. The chips were then rinsed with deionized water and dried under a stream of N₂. Oligonucleotide densities of 10¹² molecules/cm² were readily attained, as determined by carrying out the same attachment chemistry using oligonucleotide 27, with the same nucleotide sequence as oligo 28, but having in addition a Cyanine 3 fluorescent tag (Cy3) at the 3′ end. The fluorescence signal was quantified using a digital camera and compared to a calibration curve using known densities of fluorescent tagged oligonucleotides deposited on the chip surface.

### Example 5

### Attachment of Oligonucleotides to Silica Chip Surfaces Using Succinimidyl-6-hydrazinonicotinate acetone hydrazone (SAN H) Chemistry

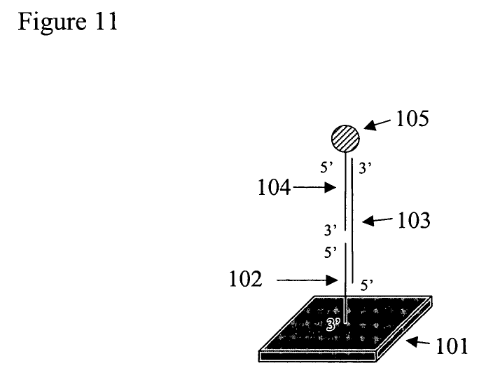

An alternative route to surface-attached oligonucleotides involves making the SANH derivative of a 3′-amino terminated oligonucleotide. SANH was purchased from Solulink (San Diego, Calif.). Attachment through the 3′ end is required for single-stranded analyte DNA to be detected through bridging an oligonucleotide probe bound to laccase and the oligonucleotide probe bound to the chip surface as diagrammed in FIG. 11. In this diagram, 101 is the silica chip, 102 is the oligonucleotide probe with its 3′ end attached to the silica chip surface, 103 is single stranded analyte DNA, 104 is an oligonucleotide probe attached to laccase, which is 105. The polarity of the oligonucleotides and single stranded analyte DNA are shown using 5′ and 3′ designations.

SANH-Derivatized Oligonucleotide

Oligonucleotides 51 and 53 (SEQ ID NO:3), each with the same sequence and a 3′-amine, but oligo 53 additionally having a 5′ Cy3 tag, were purchased from Integrated DNA Technologies, Inc. (Coralville, Iowa). To 10 μL of 3′-amine-terminated oligonucleotide in H₂O (1 mM) were added 2 μL of 0.5 M NaHPO₄, pH 7.4. Then DMF (0.6 μL) was added as was 4.33 μL SANH in DMF (solution: 1 mg SANH in 100 μL DMF). The total of 17 μL was incubated for 3 h at room temperature. After incubation, the reaction mix was diluted to 500 μL with H₂O and spun in a Biomax 5 spin filter. The sample was washed two more times with water to remove unreacted SANH and stored at −80° C.

Attachment to Surface

Silane-treated chips were prepared as described in Example 8 above. The chips were then incubated for 2 h, at room temperature with 10 μM SANH-derivatized oligonucleotide 51 or 53, each dissolved in 100 mM Na Acetate pH 5.0. Incubation was carried out in Petri plates maintained at 100% relative humidity. Following incubation, the chips were rinsed with H₂O and dried under a stream of N₂.

The fluorescence signal arising from oligo 53, attached to the chip surface, was quantified using a digital camera and compared to a calibration curve using known densities of fluorescent tagged oligonucleotides deposited on a chip surface. The surface density of oligo 53 was on the order of 10¹³ molecules per cm².

To increase the specificity of hybridization, the chips were incubated with Sigma #6429 1×Blocking buffer for 1 h. Excess blocking solution was removed by rinsing with 50 mM Na Acetate pH 5.0 plus 100 mM Na₂SO₄. Surface-bound oligonucleotide 51 was hybridized separately to:
1) Oligonucleotide 52 (SEQ IND NO:4), acting as a target sequence, hybridized to oligonucleotide 54 (SEQ IND NO:5). Oligonucleotide 54 has 18 nucleotides complementary to the 3′ end of the target oligonucleotide 52 and a fluorescent tag, Alexa Fluor 546, at its 5′ end. Oligonucleotide 52 has at its 5′ end 18 nucleotides complementary to oligonucleotide 51.
2) Oligonucleotide 52 (SEQ IND NO:4), acting as a target sequence, hybridized to oligonucleotide 50 (SEQ IND NO:5). Oligonucleotide 50 has the same nucleotide sequence as oligonucleotide 54 and was attached to laccase enzyme (lac/oligo 50) as described below.

This oligonucleotide also has a Cy3 tag at its 3′ end.

All oligonucleotides were purchased from Integrated DNA Technologies, Inc. (Coralville, Iowa). Oligos 52 and 54 in (1), and oligos 52 and 50 in (2) were prehybridized in 50 mM Na Acetate pH 5.0 plus 100 mM Na₂SO₄ for 10 min at 50° C., then allowed to cool to room temperature before adding to the chip with oligo 51.

Oligo 50 was attached to laccase using aldehyde-hydrazide attachment chemistry. Laccase from Trametes versicolor (6.2 μM; Wacker Chemie GmbH, Munich, Germany) was treated with NaIO₄ (120 mM) in 100 mM Na Acetate pH 5 for 1 h at room temperature. Ethylene glycol (120 mM) was then added to quench the unreacted periodate. The oxidized laccase was washed twice by diluting in 100 mM Na Acetate pH 5.0 and concentrating in an Amicon Ultra-4 (30,000 MWCO) (Millipore; Billerica, Mass.). A third wash was carried out in 0.5 M NaPO₄, pH 7.4. Overall wash was 1600-fold.

Periodate-treated laccase (32.5 μM) was treated with 325 μM of oligonucleotides 50, which has a hydrazide group at the 5′ end (C6 I-link, Integrated DNA Technologies, Inc.) in 200 μL for 1 h at room temperature. Two μL of 5 M NaBH₃CN in 1 M NaOH was added (final concentration 50 mM) and incubated at room temperature for 30 min. Unreacted aldehyde sites were then removed by the addition of 1 μL of 3 M ethanolamine, pH 7 (final concentration 15 mM) followed by 30 min incubation at room temperature. The laccase was then washed 3-times in an Amicon Ultra-4 (30,000 MWCO) (Millipore) with 100 mM Na Acetate pH 5.0. Total wash 8000-fold.

The resulting preparation was analyzed by SDS-polyacrylamide gel electrophoresis. Staining the protein bands with Coomassie Blue showed that the molecular mass of the laccase was increased by this treatment. Staining of parallel lanes on the same gel with ethydium bromide revealed that the same protein bands also contained oligonucleotides. Both indicated that the oligonucleotides were indeed attached to laccase.

Hybridizations of the surface bound probe, oligo 51, with the samples listed above were carried out in 50 mM Na Acetate pH 5.0 plus 100 mM Na₂SO₄. Unbound oligonucleotides were removed by rinsing with Washing Buffer A (Arraylt, Telechem International Inc., Sunnyvale, Calif.). Where a laccase/DNA adduct was used for hybridization, 50 mM Na Acetate pH 5.0 plus 100 mM Na₂SO₄ was used for the wash. After washing, the chip was covered with a cover slip and observed using a fluorescence microscope. The extent of hybridization was evaluated by measuring fluorescence emission from fluorescent-tagged oligonucleotides attached or not to laccase.

Results of fluorescent signals following hybridizations in 1) and 2) above, using different concentrations of oligos as listed, are given in Table 1. Controls of non-complementary oligo hybridizations and incubation under hybridization conditions with periodate-treated laccase labeled directly with Alexa Fluor 555 hydrazide were close to background levels. The fluorescence signals for both hybridizations 1) and 2) were dependent upon added oligo concentration, indicating that the target oligo did hybridize to the surface-attached oligo 51, and that both the Alexa Fluor-labeled oligo 54 and the laccase-coupled oligo 50 were hybridized to the free end of the hybridized target oligo 52. Thus the capture moiety (oligo 51) did capture the target analyte (oligo 52) and an analyte receptor (oligo 50) in the reporter conjugate (lac/oligo50) did bind to the target analyte.

TABLE 1
Fluorescence signals following hybridization to surface bound
oligo.

| | | Relative |
|---|---|---|
| | Camera | signal |
| Concentration | exposure | amplitude |
| 1 nM Oligo 52 + Oligo 54 | 30 s | 39, 50 |
| 10 nM Oligo 52 + Oligo 54 | 30 s | 940, 990 |
| 100 nM Oligo 52 + Oligo 54 | 30 s | 1140, 1690 |
| 1 nM Oligo 52 + Lac/Oligo 50 | 30 s | 100 |
| 10 nM Oligo 52 + Lac/Oligo 50 | 30 s | 180 |
| 100 nM Oligo 52 + Lac/Oligo 50 | 30 s | 200, 350 |

Hybridizations were also performed as in (1) except that analyte oligo 52 was added to the surface attached oligo 51 first, followed by hybridization to fluorexcent-tagged oligo 54. Similar results to those shown above were obtained.

### Example 6

### Attachment of DNA to Surfaces of CNT Device

The silica surfaces of two chips containing, on their surfaces, carbon nanotube devices were coated first with triethoxy-silylbutyraldehyde by vapor phase deposition in vacuo for 5.5 h. The chips were then either heated in a vacuum oven for 1 h at 100° C. or left overnight under dry N₂. Two perfectly complementary oligonucleotides, oligo 28 and oligo 29 (SEQ ID NOs:1 and 2, respectively), each bearing a 5′ hydrazide group (I-link, Integrated DNA Technologies, Inc., Coralville, Iowa), were dissolved separately at 50 μM in 100 mM sodium acetate, pH 5.5. Ten μL of oligo 28 solution were placed in the region of one chip where the CNT devices were located and 10 μL of oligo 29 solution were placed in the region of a second chip where the CNT devices were located, and both were allowed to incubate for 45 min at room temperature under conditions of 100% relative humidity. The devices were rinsed with deionized water, dried under a stream of N₂, and treated for 15 min with 25 mM NaBH₃CN plus 60 mM ethanolamine in 50 mM NaHPO₄, pH7.4. The chips were again rinsed with dionized water, dried under a stream of N₂ and stored overnight in a sealed Petri dish containing a moistened towel at 40° C.

### Example 7

### Polymer Stamp as Separate Capture Moiety Surface

The use of a polymer stamp in a carbon nanotube device is desirable since it allows the use of multiple polymer stamps with different attached DNA probes in the same carbon nanotube device, providing cost and time savings.

A bare polymer stamp is formed by filling a silicon, hard back photoresist master mold with a solution of PDMS (polydimethylsiloxane) and then curing it in an oven at about 80° C. The structure of the mold forms a 100 μm wide and 50 μm deep channel in the polymer stamp. After the polymer stamp is peeled away from the mold, it is processed chemically in order to modify the surface in the channel with molecules suitable for DNA attachment. A thin layer of SiO₂ is deposited onto the stamp channel surface in order to use the silane-based chemistry for DNA attachment as described in Examples 8 and 9. The polymer stamp is then incubated with a solution containing single stranded 3′-SANH-derivatized oligonucleotide probe molecules. Once attachment is complete, the stamp is washed with a buffer solution to remove unattached molecules. The polymer stamp is used in two different ways: (1) The polymer stamp is then placed on top of the carbon nanotube device and pressed against the surface of the chip to seal the channel against leakage. The channel is centered with respect to the sensing area of the device, so that a buffer solution can flow through the channel and over the carbon nanotubes. A solution containing the reporter conjugate with an oligo analyte receptor and analyte DNA is then pushed through the channel, and DNA hybridization to the surface-bound oligonucleotide probes is allowed to proceed. Unbound reporter conjugate is removed by washing and reporter substrate is added to begin the detection; or (2) before placing the polymer stamp on the chip, it is incubated in a solution of reporter conjugate with an oligo analyte receptor and analyte DNA to allow hybridization to the surface-attached oligonucleotide probes. Unbound reporter conjugate is removed by washing. The polymer stamp is then centered and placed on top of the carbon nanotube device and pressed against its surface as above. The reporter substrate solution is then flowed into the channel for the detection of bound laccase. The latter approach has the advantage of hybridization taking place away from the CNT device making the test simple and easily repeatable, and allowing reuse of devices.

What is claimed is:

1. A nanosensor for detecting the presence of an analyte comprising:
a) at least two electrodes connected by an electrically conducting path comprised of one or more carbon nanotubes wherein at least one of said carbon nanotubes is semiconducting and wherein the carbon nanotube is in contact with an effector;
b) a capture moiety having affinity for an analyte and attached to a surface;
c) a reporter conjugate comprising a reporter molecule linked to an analyte receptor, said analyte receptor having affinity for the analyte; and
d) a reporter substrate.

2. A nanosensor for detecting the presence of an analyte comprising:
a) at least two electrodes connected by an electrically conducting path comprised of one or more carbon nanotubes wherein at least one of said carbon nanotubes is semiconducting and wherein the carbon nanotube is in contact with an effector;
b) a capture moiety having affinity for a catalytic analyte and attached to a surface; and
c) a reporter substrate.

3. A nanosensor for detecting the presence of an analyte comprising:
a) at least two electrodes connected by an electrically conducting path comprised of one or more carbon nanotubes wherein at least one of said carbon nanotubes is semiconducting and wherein the carbon nanotube is in contact with an effector; and
b) a reporter molecule attached to a surface.

4. A nanosensor according to any of claims 1, 2, or 3 optionally comprising a gate electrode.

5. A nanosensor according to any of claims 1, 2, or 3 wherein said surface is the surface of a nanotube.

6. A nanosensor according to any of claims 1, 2, or 3 wherein said surface is the surface of a support.

7. A nanosensor according to claim 6 wherein the support is comprised of materials selected from the group consisting of silicon, silicon dioxide, silicon nitride, polysilicon, polymeric materials, glass, agarose, carbon, metals, ferromagnetic materials, nitrocellulose, nylon, insulating materials and semiconducting materials.

8. A nanosensor according to claim 6 wherein said support is in the form of a bead.

9. A nanosensor according to claim 6 wherein said support is in the form of a pad or stamp.

10. A nanosensor according to claim 9 wherein said stamp comprise a channel.

11. A nanosensor according to claim 1 or 2 wherein the capture moiety is a first member of a binding pair and the analyte is a second member of a binding pair.

12. A nanosensor according to claim 1 wherein the analyte receptor is a first member of a binding pair and the analyte is a second member of a binding pair.

13. A nanosensor according to claim 11 or 12 wherein the first and second members of a binding pair are members of binding pairs selected from the group consisting of antigen/epitope, receptor/ligand, binding protein/protein, nucleic acid binding polypeptide/nucleic acid, and complementary nucleic acid single strands.

14. A nanosensor according to any of claims 1, 2, or 3 wherein the carbon nanotube is supported on a support.

15. A nanosensor according to any of claims 1, 2 or 3 wherein the carbon nanotube is suspended between at least two electrodes.

16. A nanosensor according to any of claims 1, 2, or 3 wherein the effector is selected from the group consisting of oxygen, ammonia, nitrogen dioxide, and hydrogen ions.

17. A nanosensor according to claim 14 wherein the support is comprised of materials selected from the group consisting of silicon, polysilicon, silicon dioxide, silicon nitride, polymeric materials, glass, agarose, nitrocellulose, nylon, and insulating materials.

18. A nanosensor according to claim 1 or 3 wherein the reporter molecule is an enzyme.

19. A nanosensor according to claim 18 wherein the enzyme is selected from the group consisting of glucose oxidase, laccase, ascorbate oxidase, alphahydroxy acid oxidase, aldehyde oxidase, L-amino acid oxidase, ascorbate oxidase, cholesterol oxidase, bilirubin oxidase, xanthine oxidase, glutaminase, and asparginase.

20. A nanosensor according to claim 1 or 2 wherein the reporter substrate is selected from the group consisting of glucose, ascorbate, glutamine, and asparagine.

21. A nanosensor according to any of claims 1, 2, or 3 wherein the carbon nanotube is uncoated and substantially free of metal.

22. A method for detecting an analyte comprising:
a) providing a nanosensor comprising:
i) at least two electrodes connected by an electrically conducting path comprised of one or more carbon nanotubes wherein at least one of said carbon nanotubes is semiconducting, and wherein the carbon nanotube is in contact with an effector molecule and has a baseline conductance;
ii) a capture moiety having affinity for an analyte, the capture moiety attached to a surface; and
iii) a reporter conjugate comprising an analyte receptor and a reporter molecule;
b) providing a sample suspected of containing an analyte;
c) contacting the sample of (b) with the capture moiety of the nanosensor of (a) wherein the analyte present in the sample binds to the capture moiety and the analyte receptor of the reporter conjugate to form a capture-analyte-reporter complex;
d) contacting the capture-analyte-reporter complex of step (c ) with a reporter substrate wherein the concentration of the effector is altered resulting in a change in the conductance of the carbon nanotube with respect to the baseline conductance; and
e) measuring the change in the conductance of the carbon nanotube with respect to the baseline conductance whereby the presence of the analyte is detected.

23. A method for detecting a catalytic analyte comprising:
a) providing a nanosensor comprising:
i) at least two electrodes connected by an electrically conducting path comprised of one or more carbon nanotubes wherein at least one of said carbon nanotubes is semiconducting, and wherein the carbon nanotube is in contact with an effector molecule and has a baseline conductance; and
ii) a capture moiety having affinity for an analyte, the capture moiety attached to a surface;
b) providing a sample suspected of containing an catalytic analyte;
c) contacting the sample of (b) with the capture moiety of the nanosensor of (a) wherein the catalytic analyte present in the sample binds to the capture moiety to form a capture-analyte complex;
d) contacting the capture-analyte complex of step (c ) with a reporter substrate wherein the concentration of the effector molecule is altered resulting in a change in the conductance of the carbon nanotube with respect to the baseline conductance; and
e) measuring the change in the conductance of the carbon nanotube with respect to the baseline conductance whereby the presence of the analyte is detected.

24. A method for detecting an analyte comprising:
a) providing a nanosensor comprising:
i) at least two electrodes connected by an electrically conducting path comprised of one or more carbon nanotubes wherein at least one of said carbon nanotubes is semiconducting, and wherein the carbon nanotube is in contact with an effector molecule and has a baseline conductance; and
ii) a reporter molecule having an analyte substrate, the reporter molecule attached to a surface;
b) providing a sample suspected of containing an analyte substrate wherein the concentration of the effector molecule is altered resulting in a change in the conductance of the carbon nanotube with respect to the baseline conductance; and
c) measuring the change in the conductance of the carbon nanotube with respect to the baseline conductance whereby the presence of the analyte is detected.

25. A method according to either of claims 22 or 23 wherein the capture moiety is a first member of a binding pair and the analyte is a second member of a binding pair.

26. A method according to claims 22 wherein the analyte receptor is a first member of a binding pair and the analyte is a second member of a binding pair.

27. A method according to claim 25 or 26 wherein the first and second members of a binding pair are members of binding pairs selected from the group consisting of antigen/epitope, receptor/ligand, binding protein/protein, nucleic acid binding polypeptide/nucleic acid, and complementary nucleic acid single strands.

28. A method according to any of claims 22, 23, or 24 wherein the effector is selected from the group consisting of oxygen, ammonia, nitrogen dioxide, and hydrogen ions.

29. A method according to any of claims 22, 23, or 24 wherein said surface is that of a carbon nanotube.

30. A method according to any of claims 22, 23, or 24 wherein said surface is that of a support.

31. A method according to any of claim 30 wherein said support is comprised of materials selected from the group consisting of silicon, silicon dioxide, silicon nitride, polysilicon, polymeric materials, glass, agarose, carbon, metals, ferromagnetic materials, nitrocellulose, nylon, insulating materials and semiconducting materials.

32. A method according to claim 22 or 23 wherein the reporter molecule is an enzyme.

33. A method according to claim 22 wherein the reporter or the reporter conjugate is an enzyme which is activated upon binding of the analyte by the reporter conjugate.

34. A method according to claim 23 wherein the analyte is an enzyme.

35. A method according to claim 32 or 33 wherein the enzyme is selected from the group consisting of glucose oxidase, laccase, ascorbate oxidase glutaminase, and asparginase.

36. A method according to either of claims 22 or 23 wherein the reporter substrate is selected from the group consisting of glucose, ascorbate, glutamine, and asparagine.

37. A method according to claim 22 wherein the analyte is selected from the group consisting of a nucleic acid, a polypeptide, a virus, a cell, a metabolite and a product.

38. A nanosensor for the detection of an analyte comprising:
a) at least two electrodes connected by an electrically conducting path comprised of one or more carbon nanotubes wherein at least one of said carbon nanotubes is semiconducting and wherein the carbon nanotube is in contact with an effector;
b) a means for altering the concentration of said effector in response to the presence of an analyte.

39. A method for detecting the presence of an analyte comprising:
a) providing a nanosensor comprising:
i) at least two electrodes connected by an electrically conducting path comprised of one or more carbon nanotubes wherein at least one of said carbon nanotubes is semiconducting and wherein the carbon nanotube is in contact with an effector and wherein the carbon nanotube has a baseline conductance;
ii) a means for altering the concentration of said effector in response to the presence of an analyte;
b) contacting the nanosensor of (a) with an analyte whereby the concentration of said effector is altered resulting in a change from the baseline conductance of said carbon nantube; and
c) measuring the alteration from the baseline conductance of the carbon nanotube of (b) wherein the presence of the analyte is detected.


SEQUENCE LISTING (5 sequences)

SEQ ID NO: 1 - DNA, 18 bp, artificial sequence
  synthetic oligo
cgctgtgatggtggcccc

SEQ ID NO: 2 - DNA, 18 bp, artificial sequence
  synthetic oligo
ggggccaccatcacagcg

SEQ ID NO: 3 - DNA, 28 bp, artificial sequence
  synthetic oligo
cgctgtgatggtggcccctttttttttt

SEQ ID NO: 4 - DNA, 46 bp, artificial sequence
  synthetic oligo
ggggccaccatcacagcgttttttttttggggccaccatcacagcg

SEQ ID NO: 5 - DNA, 18 bp, artificial sequence
  synthetic oligo
cgctgtgatggtggcccc